(19) United States
(12) Patent Application Publication
(10) Pub. No.: US 2023/0273185 A1
(43) Pub. Date: Aug. 31, 2023

(54) ELECTRODE INTEGRATED MICROSIEVE ASSEMBLY

(71) Applicant: TECHNISCHE UNIVERSITEIT EINDHOVEN, EINDHOVEN (NL)

(72) Inventors: Regina LÜTTGE, EINDHOVEN (NL); Yagmur Demircan YALCIN, EINDHOVEN (NL)

(21) Appl. No.: 18/017,891

(22) PCT Filed: Jul. 27, 2021
(86) PCT No.: PCT/EP2021/071030

§ 371 Date: Jan. 25, 2023

Related U.S. Application Data
(60) Provisional application No. 63/056,809, filed on Jul. 27, 2020.

(51) Int. Cl. G01N 33/487; B01L 3/00

(52) U.S. Cl. CPC G01N 33/48728; G01N 33/48785; B01L 3/502715; B01L 2300/0645; B01L 2300/0829

(57) ABSTRACT

The invention relates to a device for detecting and/or characterizing one or more cells by the electrical properties of the cells, the device comprising at least one electrode integrated microsieve assembly, wherein the assembly comprises a) a microsieve arrangement, such as a microsieve array, comprising one or more micropores for retaining the cells, and b) a substrate comprising one or more pairs of oppositely arranged first and second electrodes, wherein the microsieve arrangement is connected to the substrate such that each of the one or more pairs of electrodes is configured to form an electric field in at least one micropore of the microsieve arrangement, and wherein the first electrode is arranged in parallel to the second electrode.

### TECHNICAL FIELD

The present invention relates to a device for detecting and/or characterizing a cell by the electrical properties of said cell. Moreover, the present invention relates to a microsieve arrangement comprising one or more micropores. The present invention also relates to a substrate comprising one or more pairs of first and second electrodes. The present invention also relates to a method for detecting and/or characterizing a cell by the electrical properties of said cell and to a use of an arrangement for determining a deflection of a cell.

### BACKGROUND

Microsieve arrangements, such as microsieve arrays, have typically been developed for use in various biotechnology applications, such as in vitro diagnostics, cell isolation assays, cell culturing assays, immunoassays and in vivo-like cell culturing assays (e.g. organ-on-a-chip). In microsieve-based assays one or more cells (e.g. a cell population) is contacted with a microsieve arrangement, such as a microsieve array, i.e. an artificial device comprising one or more micropores. Microsieve-based assays have been found to provide elaborate understanding of intra- and extracellular mechanisms and cellular interactions in specific tissue formations, even at a single cell level. In the case of a microsieve arrangement a hydrodynamic flow can be used to trap (retain) a cell or a cell population, even a single cell, inside a micropore. Once a cell is retained in said micropore, the hydrodynamic resistance prevents that another cell can be retained in the same micropore. For a complete overview of in vitro assays reference is made to Tietz textbook of clinical chemistry and molecular diagnostics (Connell, 2012, 5^{th} edition).

Generally, microsieve-based assays are used in conjunction with optical microscopy. However, in the application example of pharmaceutical screening, it is cumbersome and costly to use per cultured sample optical microscopy to inspect yield of cell seeding in the microsieve pores and monitor their position and behaviour during, for example, a stem cell differentiation process by means of the same. Moreover, dyes used to make monitoring easier are toxic (Pan, Y., Hu, N., Wei, X., Gong, L., Zhang, B., Wan, H., Wang, P., 2019. 3D cell-based biosensor for cell viability and drug assessment by 3D electric cell/matrigel-substrate impedance sensing. Biosens. Bioelectron. 130, 344-351) for biological cells. Therefore, after this analysis, cells can die, and the assay should be ceased. As an alternative to the use of optical microscopy, one solution is to integrate thin-film electrodes on the sidewalls of the micropores of such microsieve arrangement to detect and/or characterize cell activity and couple these to contact lines in a similar fashion as a planar microelectrode array (MEA) configuration.

Such microelectromechanical systems (MEMS)-based devices, combining microsieve arrangements with microelectrode arrays (MEAs), enable the read-out of signals of a cell population, even at a single cell level. In conventional strategies for electrode integration with cell culture arrangements, silicon microsieve substrates are used. While potentially very informative and rich data can be collected from such silicon-based integrated constructs, however the costs involved in using these conventional substrates, i.e. silicon microsieves having thin-film electrodes integrated on the sidewalls of micropores, in a pharmaceutical screening application are high and limit progress on fundamental research and pharmaceutical development of new drugs. In addition, fabrication of silicon microsieves having oppositely positioned thin-film electrodes integrated on the sidewalls of micropores is challenging and yield of manufacturing is low. Also the re-usability of said microsieves having thin-film electrodes integrated on the sidewalls of micropores is low. The thin-film sidewall electrodes are in contact with a cell or cell population and need to be rigorously cleaned prior to re-use. This lack of re-usability limits progress on fundamental research and pharmaceutical screening applications for, for example, new drugs.

In addition to the use of silicon microsieve substrates a more cost-effective polymer microsieve substrate for cell culture experiments has been developed (E. Moonen, R. Luttge, and J. P. Frimat, Microelectron. Eng. 197, 1 (2018)). Said microsieve substrates made entirely from polymers (e.g. plastics, Norland Optical Adhesive 81 (“NOA81”) and the like) were shown to have far lower stiffness than silicon and hence serve a much better and cost-effective measuring strategy. However, the integration of conventional oppositely positioned thin-film electrodes on the sidewalls of the micropores of such a polymer-based microsieve arrangement proved to be not so straightforward as for silicon microsieves. Hence, there is a need for providing novel strategies for integrating electrodes, suitable for detecting and/or characterizing cell activity, with a polymer-based microsieve comprising micropores.

### Definitions

A portion of this disclosure contains material that is subject to copyright protection (such as, but not limited to, diagrams, device photographs, or any other aspects of this submission for which copyright protection is or may be available in any jurisdiction). The copyright owner has no objection to the facsimile reproduction by anyone of the patent document or patent disclosure, as it appears in the Patent Office patent file or records, but otherwise reserves all copyright rights whatsoever.

Various terms relating to the methods, compositions, uses and other aspects of the present invention are used throughout the specification and claims. Such terms are to be given their ordinary meaning in the art to which the invention pertains, unless otherwise indicated. Other specifically defined terms are to be construed in a manner consistent with the definition provided herein. Although any methods and materials similar or equivalent to those described herein can be used in the practice for testing of the present invention, the preferred materials and methods are described herein.

“2D”: The term 2D, also referred to as two-dimensional, refers to the geometric setting of an object, wherein the object is defined as having two dimensions expressed in the two parameters of length and width. Commonly, an object in 2D is in the form of a flat surface i.e. a planar object for example a circle, square, rectangle etcetera.

“3D”: The term 3D, also referred to as three-dimensional, refers to the geometric setting of an object, wherein the object is defined as having three dimensions expressed in any combination of three from the parameters comprising: width, height, depth, and length. As used herein a 3D dimension may also be expressed by diameter (i.e. width and length) and height of an object or by variations of such a base geometry and height of an object. For example, a 3D pore can have a diameter of 100 μm and a height of 100 μm, giving it a 3D dimension e.g. in the form of a cylinder. Examples of three-dimensional objects are a cube, a cylinder, a cone, a cuboid, a pyramid etcetera.

“A”, “an,” and “the”: these singular form terms may include plural referents unless the content clearly dictates otherwise. Thus, for example, reference to “a cell” includes a combination of two or more cells, and the like.

“About” and “approximately”: these terms, when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

“And/or”: the term “and/or” refers to a situation wherein one or more of the stated cases may occur, alone or in combination with at least one of the stated cases, up to with all the stated cases.

“Array”: the term “array” refers to a systematic arrangement of similar objects, usually in rows and columns. The term as used herein is used interchangeably with the terms “arrangement” and “substrate”. In biotechnological sciences the term “array” is commonly used to refer to a multiplex lab-on-a-chip (i.e. an object suitable for performing multiple experimental measurements about simultaneously). An array as used herein comprises a microarray, i.e. a miniaturized, multiplexed lab-on-a-chip.

“Assembly”: this term in general refers to the combination of multiple components into an assembled object. As used herein “assembly” is used to refer to the combination of an “arrangement”, such as an array, construed such that it is plugged onto a “substrate”.

“Comprising”: this term is construed as being inclusive and open ended, and not exclusive. Specifically, the term and variations thereof mean the specified features, steps or components are included. These terms are not to be interpreted to exclude the presence of other features, steps or components.

“Electrode”: this term, used interchangeably with “electrical conductor” throughout the disclosure, refers to an object comprising a material suitable for allowing the flow of an electrical current in one or more directions. Specifically, the term and variations thereof as used herein refer to electrodes and/or electrical conductors suitable for use in micro electromechanical systems (MEMS) such as, microelectrode arrays (MEAs).

“Micropore”: the term “micropore” as used herein refers to an opening or aperture having appropriate dimensions (e.g. diameter) for retaining one or more cells in a media.

“Microsieve”: the term “microsieve” as used herein refers to an arrangement, such as a microsieve array, comprising one or more micropores. Said microsieve may comprise multiple micropores that are about uniform in size and are about evenly distributed on the microsieve.

“Substrate”: the term “substrate” as used herein refers to a planar layer of a substance or material that is underlying to another material, for example underlying to an arrangement as part of a device. As used herein a substrate is a part of an assembly or device. As used herein a substrate comprises a systematic arrangement of similar objects, such as electrodes, usually in rows and columns and therefore as used herein may be interchangeably used with the term “array”.

“System”: the term “system” as used herein also refers to an assembly or to a device comprising one or more assemblies, for example to a device comprising electrode integrated microsieve assembly. The system may herein also be referred to as a platform.

### SUMMARY OF THE INVENTION

Given the electrode integrated microsieve assemblies described in the art, the inventors developed a novel device for detecting and/or characterizing of one or more cells by the electrical properties of said cells. The invention provides hereto a device for detecting and/or characterizing one or more cells by the electrical properties of said cells, the device comprising at least one electrode integrated microsieve assembly, wherein the assembly comprises a) a microsieve arrangement, such as a microsieve array, comprising one or more micropores for retaining said cells, and b) a substrate comprising one or more pairs of oppositely arranged first and second electrodes, wherein the microsieve arrangement is connected to the substrate such that each of the one or more pairs of oppositely arranged electrodes is configured to form an electric field in at least one micropore of the microsieve arrangement, and wherein said first electrode is arranged in parallel to said second electrode.

By means of “in parallel” as used herein is meant that a first electrode of a pair of a first electrode and second electrode is arranged oppositely to the second electrode such that the distance between the first electrode is about the same all along the length of the first electrode to the second electrode. It is contemplated that a slight variation in distance over the entire surface area is allowable given the technical implications of a molding and/or remolding process for the substrate. As such, the term “in parallel” allows for a slight variation in distance over the entire surface area and may thus comprise that a first electrode is arranged nearly parallel, or about parallel, to a second electrode. It is noted that the one or more pairs of oppositely arranged first and second electrodes preferably extends substantially perpendicular from the substrate.

In particular the present invention provides a device wherein the microsieve arrangement is detachably connected to the substrate.

It was found that by means of the device of the present invention the microsieve arrangement can be utilized as a disposable add-on for a re-usable electrode-comprising substrate by carrying out a passively aligned click-on hybrid assembly step. In other words, the microsieve arrangement is arranged such that it is pluggable with the electrode-comprising substrate.

It was found that said device makes the use of expensive integrated silicon micromachining and thin-film technology for the fabrication of electrically functionalized microsieves, i.e. silicon microsieves having thin-film electrodes integrated on the sidewalls of micropores, largely redundant.

It was further surprisingly found that the device according to the present invention was able to detect and/or characterize one or more cells by the electrical properties of said cells despite a relatively thick and tapered insulating layer of polymeric material of the microsieve. In one preferred embodiment the device was able to detect a cell in between the electrical conductors via impedimetric measurements, wherein the variation amount in impendence that is caused by the presence of a cell is measured without compromising detection efficiency despite a relatively thick and tapered insulating layer of polymeric material of the microsieve.

The microsieve arrangement of the present invention may comprise one or more pairs of slots arranged such that each pair of oppositely arranged first and second electrodes can be received into a respective pair of slots. In an embodiment of the microsieve arrangement of the present invention, the first slot may be arranged at a distance of at most 75 μm, preferably at most 50 μm, more preferably about 20 μm from the second slot.

In addition, the slots of the microsieve arrangement may comprise an appropriate length, an appropriate width and an appropriate height that is arranged such that it allows for receiving a respective electrode. Preferably the slots of the present invention may have a rectangular configuration. In the case of such rectangular configuration the slots may have a depth of at most 70 μm, preferably at most 50 μm, more preferably about 20 μm and/or a width (as meant herein the “width” of a first slot is in parallel with the width of a second slot) of at most 70 μm, preferably at most 50 μm, more preferably about 20 μm. However it is noted that the width may also be larger than the preferred dimensions as listed above, e.g. the width of a slot may be adapted to the width of the respective micropore. The slots may have a height about equal to the thickness of the microsieve arrangement. For example, the slots may have a height of at most 200 μm, preferably about 100 μm.

The substrate of the present invention comprises one or more pairs of first and second electrodes suitable for use in a device for detecting and/or characterizing one or more cells by the electrical properties of said cells. According to the present invention the first electrode of one pair of electrodes is arranged in parallel to the second electrode of the same pair of electrodes. The pair of electrodes may be arranged at a predefined distance from each other, wherein the first electrode may be arranged at a distance of at most 75 μm, preferably at most 50 μm, more preferably about 20 μm from the second electrode. The electrodes may have a height about equal to the thickness of the microsieve arrangement. For example, the first and second electrodes of each pair may have a height of at most 200 μm, preferably about 100 μm. The first and second electrodes may have a depth of at most 70 μm, preferably at most 50 μm, more preferably about 20 μm and/or a width (as meant herein the “width” of a first electrode is in parallel with the width of a second electrode) of at most 70 μm, preferably at most 50 μm, more preferably about 20 μm. The electrodes of the substrate of the present invention may be fabricated of a conductive material. Preferably said material is one suitable for forming good electrical conductors. The conductive material is preferably a metal, wherein said metal preferably is selected from the group consisting of copper, zinc, nickel, lead, mercury, silver, zinc, aluminum, gold, iron or alloy comprising any one of said metals.

Further, the substrate of the present invention comprising the electrodes may comprise a hole, wherein said hole comprises a diameter of at most 10 μm, preferably about 3 μm, and wherein said hole is arranged such that it is between a pair of first and second electrodes.

The present invention further relates to a microsieve arrangement comprising one or more micropores, preferably in the form of a prism (or other suitable form), for retaining one or more cells for use in an arrangement for detecting and/or characterizing one or more cells by the electrical properties of said cells. In an embodiment of the present invention the micropore may be configured to comprise a top-opening and a bottom-opening. In case of such configuration the top-opening of said one or more micropores may have a larger opening than the bottom-opening of said one or more micropores. In particular, each of the micropores may comprise an appropriate width, appropriate length and an appropriate height for retaining one or more cells. In a preferred embodiment the top-opening and/or bottom-opening may have the form of a circle having a diameter of between at least 0 μm to 100 μm, preferably between about 2 μm and about 20 μm. In a further embodiment the micropore of the present invention may have a height between about at least 0 μm to 200 μm, preferably between about 5 μm to 100 μm, more preferably about 10 μm to 50 μm, for example size of a cell's soma.

The one or more micropores may be made from a material comprising a polymer. The material is dielectric, and comprises a permittivity constant of between about 1 and about 5, preferably about 2.75 and/or wherein the material comprises a polymer, preferably a silicon-based polymer, more preferably polydimethylsiloxane.

Optionally, the material of the one or more micropores may have a conductivity of at least 1×10⁻²⁵ S/m, preferably at least 1×10⁻²¹ S/m, more preferably about 1×10⁻¹⁶ S/m.

The present invention further relates to an electrode integrated microsieve assembly for detecting and/or characterizing one or more cells by the electrical properties of said cells comprising a) the microsieve arrangement according to the present invention, and b) the substrate according to the present invention.

In a further aspect of the present invention, the present invention relates to a kit of parts for detecting and/or characterizing one or more cells by the electrical properties of said cells comprising a) the microsieve arrangement according to the present invention, and b) the substrate according to the present invention.

In one aspect of the invention the one or more cells as provided herein is a cell or a cell population, preferably a mammalian cell or cell population. In a further aspect the one or more cells comprises a homogenous or heterogenous cell population. In one preferred aspect the one or more cells comprise neuronal cells and/or progenitor cells thereof. In yet a further aspect of the invention differently phased stem cell linages may be provided. Preferably the one or more cells as provided herein is a cell suitable for the cellular assay of interest.

The present invention also relates to a method for detecting and/or characterizing one or more cells by the electrical properties of said cells comprising the steps of:
a. providing the device according to the present invention;
b. providing one or more cells, preferably one or more cells in a medium;
c. supplying the cells of step b) to the device of step a);
d. determining a deflection of the electric field by said one or more cells.

The medium of step b) may comprise a medium, media or solution suitable for maintaining and/or culturing cells. Said medium may for example comprise PBS, cell culture medium (e.g. DMEM, IMDM etc) or any other suitable medium.

In a final aspect of the present invention, the invention relates to the use of the device according to the invention or the microsieve arrangement according to the invention and the substrate according to the invention in a method for detecting and/or characterizing one or more cells by the electrical properties of said cells.

### DETAILED DESCRIPTION

In neuronal cell culture, the spatial distribution of neurons will influence the design of the established network connections among the neurons. While arrangement of neurons as of seeding can in principle be controlled by using a microsieve array, it takes about 3-7 Days-in-Vitro (DIV) prior to outgrowth forms and connections establish amongst the seeded neurons by neural differentiation processes. When such ordered layout of neuronal cell culture is used in pharmaceutical screening, it is of outmost importance to know in which of the pores a neuron is located. Also it is most likely that neurons could be first seeded in the pores, but migrate over the days again out of it, to sense that a cell is located in the pore a 3D electrode integrated microsieve can be applied such as described in Example 2 as disclosed herein.

In this invention, a reusable platform containing 3D-electrodes is provided to electrically monitor cell placement distribution in microsieves.

The system (also referred to as a 3D pluggable system) has 3D electrodes integrated with microsieves, which was compared with the thin-film sidewall electrodes which touch cells in a 3D experiment platform. Although a relatively thick and tapered insulating layer exist between cells and electrode in the 3D pluggable system, an impedance variation ratio of 3.4% on a measurable based impedance of ˜59 kOhm was obtained.

In the platform of this invention, a new polymer-based microsieve design is provided that is pluggable with a re-usable 3D electrode array. This provides a cost-effective approach, which means that the microsieve can complement a re-usable 3D electrode apparatus used for the impedance measurement in a high-throughput and robust cell culture microenvironment. The re-usable 3D electrode array is designed for use as an impedance sensor array which will be re-usable for the large number of microsieve cultures that are generally needed in a biological study without any rigorous cleaning steps by means of the elimination of cell-electrode touching interfaces.

In an embodiment of the present invention, the device has two pluggable parts:
(i) disposable polymer-based microsieves; and
(ii) re-usable 3D electrodes.

The structure of the device of the present invention preferably has polymer microsieves comprising micropores in the form of a prismatic shape, optionally a conical shape or any variations and/or combinations thereof, with a top and bottom opening of 20 and 3 μm, respectively. In addition, the 100 μm thick polymer microsieve contains holes from the back side of 20 μm by 20 μm square openings to plug-in the 3D electrodes during assembly. However, other shapes and dimensions are also suitable for use in the device of the present invention.

Based on the structural design of this new (reusable) platform the principle of cell detection is based on the principle that biological cells act as dielectric particles. Therefore, they change the impedance of a solution in which they are immersed. At low frequencies, only the cell diameter determines the amount of variation of an impedance signal. At high frequencies, cell electrical properties affect impedance response. This technique, named impedance flow cytometry (IFC), is utilized in the device of the present invention to track cell movement and detect cell position on microsieve arrays by using the configuration according to the device of the present invention.

When a cell moves into or out of a microsieve pore, there will be an impedance change. The trend of impedance change will differ based on the movement direction. Therefore, if a cell goes inside the micropore of a microsieve and then moves outside of it, impedance change will have a characteristic fingerprint which can be determined by means of real-time measurements.

When cells move into or out of a microsieve pore, electrical dynamics in the pores of the hybrid (pluggable) assembly within a reusable platform containing 3D read-out electrodes will change, and therefore this motion of cells can be sensed by impedance spectroscopy without cell labelling. With a negligible delay in comparison with the velocity of cell movement, cells can be tracked via impedance-based measurement by means of impedance spectroscopy equipment. Therefore, this new platform enables the ability to sense cell movement in a real-time manner using the subsequent experimental steps:
1) connecting the measurement setup to the platform and
2) loading cells into the system with passive pumping, and
wherein during or after cell loading into that platform, electrical data is stored over time of running the assay and interpreted.

The above outlined experimental steps may further comprise the following additional steps:
3) incubating the cells for an appropriate time;
4) assembling the cell-loaded microsieve to the measurement platform;
5) taking readings over an appropriate period of time;
6) returning the microsieve to the incubation environment; and
7) revisiting steps 3-6 as often as required.
   Alternatively, cell loading is performed after assembling the microsieve arrangement to the substrate, thus offering the capability to follow cell positioning during the loading step.

By means of protecting the 3D electrode sites in the electrical platform from any contact with cells due to the assembly with a cheap and disposable polymeric microsieve as a cell culture plate the position of cells and potentially also its outgrowth direction during stem cell differentiation in the pores of the microsieves can be detected due to the configuration of the 3D electrodes being aligned to the 3D pores. To make quantitative interpretation, electric field and impedance of thin-film sidewall (prior art) and 3D electrodes (present invention) were compared. When compared to thin-film electrically integrated pores or the use of optical microscopy, this novel configuration is fast and cost-effective.

To realize such a cost-effective, fast and re-usable cell culture platform, a hybrid assembly of a re-usable 3D electrode array structure is presented herein that works with the polymer microsieve substrate as a complementary, but disposal part.

### Fabrication of Polymeric Microsieve Arrangement

To fabricate the cell culture substrate, a microfabrication process is applied to manufacture the microstructured polymeric substrate for cell capture and neuronal cell network culture, but with features at the top and the back side of the polymer substrate. Using, for example, a combination of micromolding an array of inverted pyramidal shaped cavities (closed micropores) is prepared. This is prepared for such disposable microsieve-based cell culture substrates from the top side of a polymeric substrate and at least two opposite prismatic cavities adjacent and aligned to the top cavity at the back side of the polymer precursors, for example in an aligned front to back side mode of replication using an appropriate mold insert. This preparation could include similar structures with guiding features such as corners, folds or nanostructures that lead to stimulate and guide neural processes during the differentiation of stem cells. The top cavities are subsequently treated by a means of micromachining, for example laser ablation or dry etching, to produce through-hole openings that are positioned relative to the deepest point in the cavity. Alternatively, also master molds only replicating the cavities at the backside and returning flat top sides can be further comprised with micropores by subsequent micromachining techniques depending on the desired pore shapes.

### Fabrication of 3D Electrodes

3D electrodes can be prepared by a number of lithographic techniques and metal deposition. In case electrodes are made from e.g. electroplated copper or nickel, these can also be used as a master for molding the openings at the backside of the polymeric microsieve, alignment of these openings are then an exact match with the openings in the microsieve, hence to form a pluggable assembly. In other words, openings permit ease of engagement and disengagement of two components.

The usage of the lithographically aligned electrodes as a master mold for backside openings ensures to minimize a potential air gap between the electrode wall and the polymer enclosing the 3D pore in the microsieve.

Demircan et al. (Electrophoresis 36, 1149, 2015) achieved copper electroplating by this method up to 30 μm height and the distance between electrodes was 15 μm previously at which this distance is considerably smaller than that being necessary for the pluggable platform (26 μm) described herein. This shows that 26 μm is attainable. By using thicker photoresist, 100 μm height or even higher aspect-ratios can be also obtained. Electrode dimension is minimally the same as the 3D pore side length of the microsieves at the top, like 20 μm by 20 μm. Therefore, a 5:1 aspect ratio is required. For example, with using SU8, this can be achieved since its aspect ratio is well over 5:1 (MicroChem, Prod. Datasheet 20, 4 (2000)). Additionally, there should be either a hole of 3 μm diameter in the 3D electrode bottom layer or at least a sufficiently large air gap to provide full operation of the microsieves, for example when cell-loading occurs in the assembled arrangement.

Other embodiments, further teachings and/or examples related to the invention are described in Example 1 and Example 2.

### EXAMPLES

### Example 1

### Methodology

Finite element modeling (FEM) enables to solve complex equations for complex structures numerically. In the structure, the electric field is not uniform between electrodes due to the presence of insulator layers. Therefore, the numeric solution is the most accurate way to evaluate the electric field in our system. Electric Currents interface of the AC/DC module of COMSOL Multiphysics 5.5 in the frequency domain with current conservation, electrical insulation, terminal in current type, and ground boundary conditions was used to test the electrical field strength in a pore. This interface of COMSOL solves current conservation equation based on Ohm's law and neglects inductive effects. Domain equations are as follows:

−∇((σ+Jωεrε0)∇V)=0 (1)

E=−∇V (2)

D=εrε0E (3)

where ε0, εr, and σ are the electrical permittivity of free space, relative permittivity, and conductivity, respectively. J is √−1, and ω is the angular frequency of the applied signal. V, E, and D denote the electric potential, electric field, and displacement, respectively. ∇ is the gradient operator.

Electrodes were defined as perfect conductors at the boundary of the microsieves' pores and have infinite thickness to decrease memory need and calculation time. Inner and outer boundaries of microsieves' pores were selected as thin-film sidewall and 3D electrodes, respectively. Solution domain electrical properties were chosen like phosphate buffer saline (PBS) which has 1.4 S/m conductivity with the permittivity constant of 80. Microsieve material was selected as polydimethylsiloxane, chosen from material library, having a permittivity constant of 2.75 and a conductivity of 10-16 S/m. By applying 1 A current and analyzing the system response at 10 kHz in the frequency domain, 2D and 3D solutions of sidewall and 3D electrode integrated microsieves were carried out. The system was tested at such relatively low frequency since it is enough to determine cell existence. Cell coordinates (given in FIG. 3(a-ii)) were kept constant for x and z directions while it was swept in y direction from 30 μm to 120 μm with 2 μm step size at both 2D and 3D experiments. By observing convergence plots, free triangular meshing style was chosen with 1.26 μm, 2.52 nm, and 1.1 as maximum, minimum element sizes, and element growth rate, respectively, in 2D experiments. In 3D experiment, free tetrahedral meshing was used with 4.41 μm, 189 nm, and 1.35 as maximum, minimum element sizes, and element growth rate, respectively. The software solved the system for the spatial distribution of electric potential. Electric field and impedance quantification were obtained during postprocessing by using the Results interface.

### Results

2D and 3D experiments of thin-film sidewall and 3D electrode integrated microsieves were carried out. Table 1 presents mesh properties, number of degrees of freedom values, and solution time specifically to the experiment. Impedance results show that in terms of impedance variation trend, there was no difference between 2D and 3D experiments (figures). However, the values of impedances (FIG. 3(a-iii) & (b-vi)) did not model the impedance magnitude of a real structure. Therefore, comparisons between thin-film sidewall and 3D electrode structures were carried out via 3D experiments even solution times were significantly longer.

TABLE 1
Meshing properties, number of degrees of freedom, and
solution time for 2D and 3D experiments of thin-film
sidewall and 3D electrode embedded microsieves.

| | | | Number of | |
|---|---|---|---|---|
| Experiment | Electrode | | degrees of | Solution time |
| type | type | Mesh elements | freedom | (s) |
| 2D | Sidewall | 24428 domain | 12412 | 7 |
| | | 736 boundary | | |
| | 3D | 24428 domain | 12412 | 6 |
| | | 736 boundary | | |
| 3D | Sidewall | 56655 domain | 80963 | 319 |
| | | 6652 boundary | | |
| | | 494 edge | | |
| | 3D | 56655 domain | 80963 | 278 |
| | | 6652 boundary | | |
| | | 494 edge | | |

FIG. 4 presents electric field distribution through centre line without cell (a-i and b-iii) and impedance variation for thin-film sidewall (a-ii) and 3D electrodes (b-v), respectively, while a cell, having 5 μm radius, moves from inside to outside of a microsieve pore. Electric field had more strength (˜3.4 times) and increasing trend in 3D electrodes design while it decreased in sidewall type since the distance between electrodes increases in sidewall electrodes, but insulator thickness decreases even distance between electrodes is constant in 3D ones.

To determine the amount of impedance change due to existence of a cell as an indicator for cell detection efficiency, the following formula was used.

Δ|z|=(|z|_{with cell}−|z|_{without cell})/|z|_{without cell}×100

|z| denotes impedance magnitude in this equation. The impedance magnitude variation of thin-film sidewall electrodes (FIG. 4(a-ii)) was calculated as 3.5%, having 2.7 kΩ baseline impedance, while the one calculated for 3D electrodes was 3.4% and baseline impedance (58.9 kΩ) was still in the measurable range (FIG. 4(b-iv)). These results proved that without compromising detection efficiency, the 3D electrodes can be integrated with microsieves in this pluggable, rapid, real-time, and label-free platform.

### Example 2

This study presents an electrical model of a 3D-electrode integrated microsieve for in vitro neurophysiological analysis. By demonstrating that the electric field is sufficiently high, it is possible to design choices for a reusable cell seeding monitor. The results show that there is only five times difference between the electric field of a 3D-electrode versus a thin-film integrated side-wall electrode.

Using integrated silicon micromachining and thin-film technology the fabrication of electrically functionalized inverted pyramid-shaped pores (FIG. 5a) was a major challenge and is still very expensive at the current scale of device production, which is limited to fundamental research. Also, thin-film side-wall electrodes are in contact with the neurons and the microsieves need to be rigorously cleaned prior to re-use. To simplify such microsieve studies on neuronal cell networks, analysis was started by optical techniques on polymeric microsieves, which also proved to be valuable. Knowing the distribution of cells throughout the pores of the sieve, however, will enhance statistical relevance of these biological experiments. Hence, a cost-effective, fast and reusable electrical platform to monitor cell placement distribution by containing 3D-electrodes (FIG. 5b) would be beneficial. The Electrical Currents interface of the AC/DC module of COMSOL Multiphysics 5.5 was used in the frequency domain with current conservation, electrical isolation, electrical potential, and ground boundary conditions to demonstrate electrical field strength. By applying 10Vₚ voltage at 300 Hz, the electrical field norm (taking into account x, y, and z components) was obtained for side-wall (FIG. 6a) and 3D-electrode configurations (FIG. 6b) integrated with microsieves.

The results show that 3D-electrodes have a significantly lower electrical field than that of the side-wall type up to 95 μm height of the micropore due to the thick dielectric layer but it was one-fifth between 95-100 μm (FIG. 6).

This result indicates that a neuron is detectable in the 3D electrode configuration, while moving into the micropore.

### Example 3

The impedance spectrum of 3D-electrodes has been measured by using a commercial impedance spectrometer (HF2LI-Zurich Instrument).

100% Phosphate buffer saline (PBS) (100), 50% PBS in deionized water (50), and deionized water were used as different conductivity solutions.

Impedance characteristics of 3D-electrodes has been compared as proof-of-concept (FIG. 7).

These results indicate that with this platform, i.e., 3D-electrode integrated micropores, impedance measurement can be achieved. Additionally, the difference between different conductivity solutions is sensible fora frequency range (Δf) of 1 MHz.

### DESCRIPTION OF THE DRAWINGS

FIG. 1 shows an electrode integrated microsieve assembly according to the present invention.

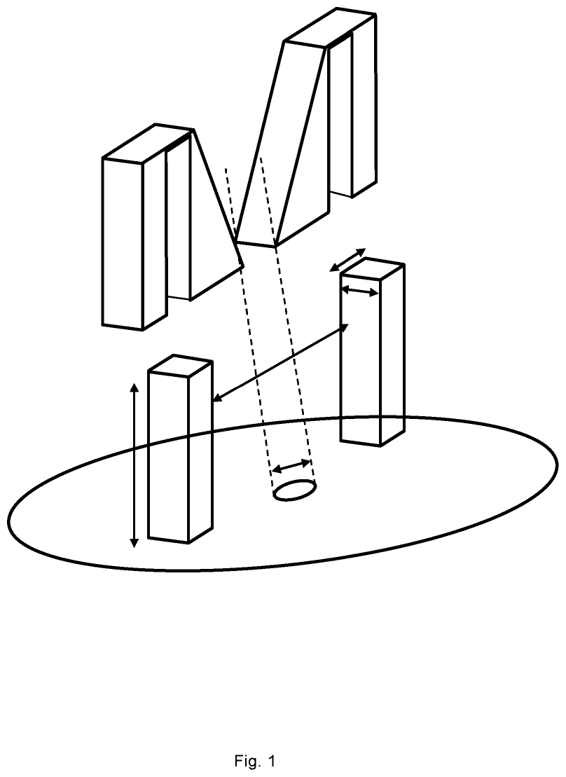

FIG. 2a shows a schematic depiction of an electrode integrated microsieve assembly according to the present invention, comprising disposable a microsieve arrangement and a re-usable 3D electrodes substrate.

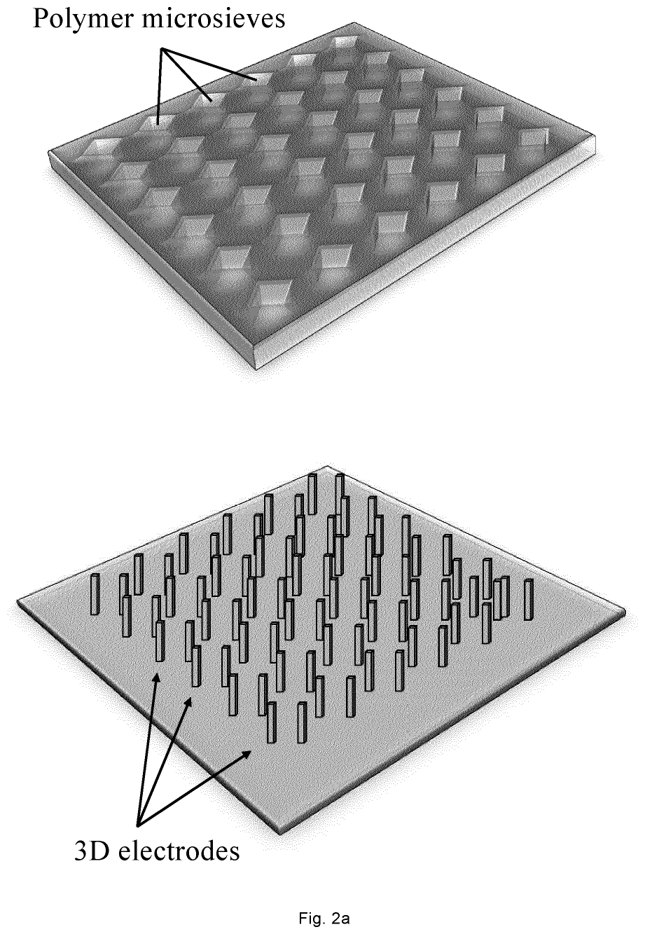

FIG. 2b shows one microsieve pore of an electrode integrated microsieve assembly according to the present invention.

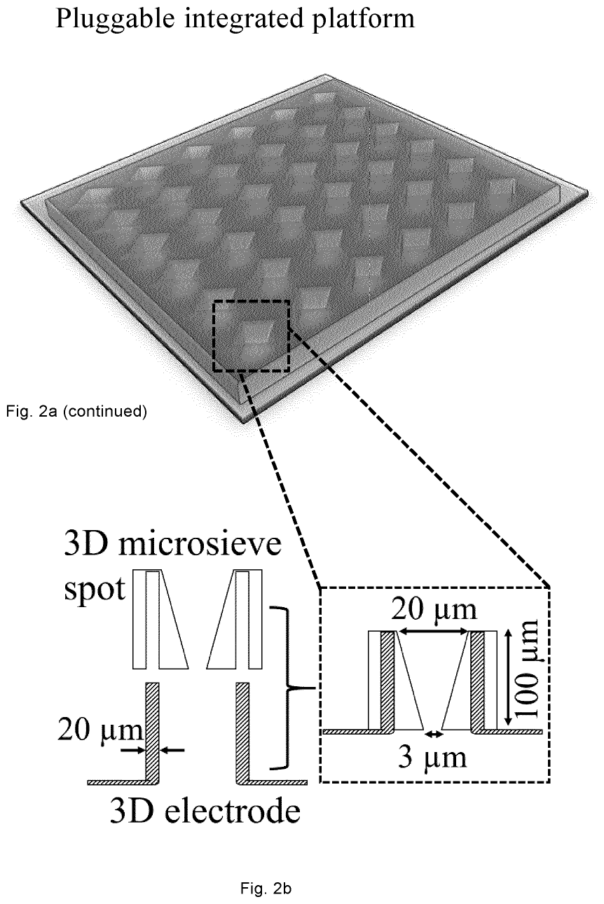

FIG. 3a shows results for electric field strength for 2D thin-film sidewall electrodes. Cell movement direction and the schematic of electrodes integrated in microsieves (3a-ii) and the variation in impedance magnitude while a cell moving in thin-film sidewall (3a-iii) are shown.

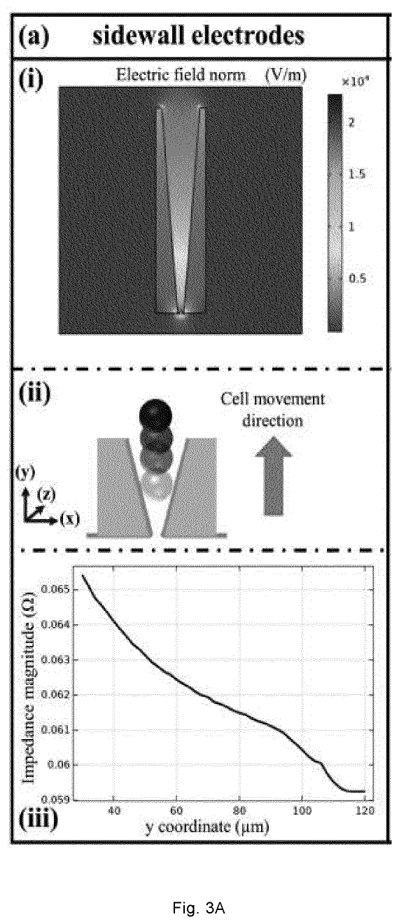

FIG. 3b shows results for electric field strength for 3D electrodes. Cell movement direction and the schematic of electrodes integrated in microsieves (3b-v) and the variation in impedance magnitude while a cell moving in 3D electrode integrated microsieve assembly (3a-vi) are shown.

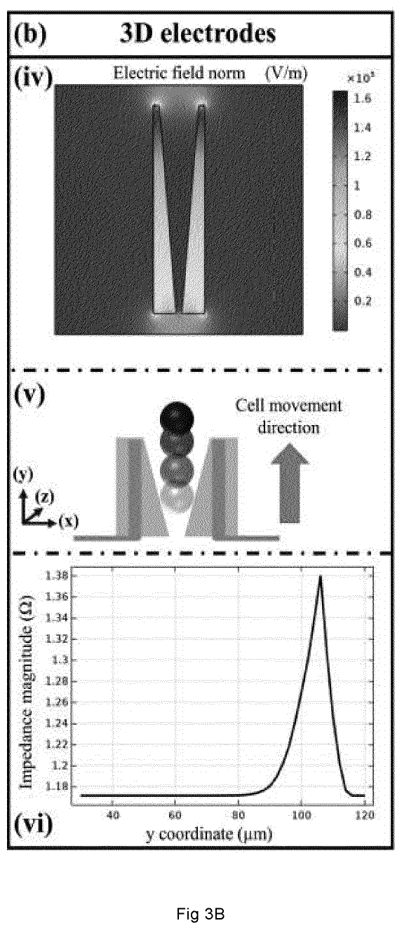

FIG. 4a shows the 3D electric field and impedance magnitude results of thin-film sidewall (4a-i and ii).

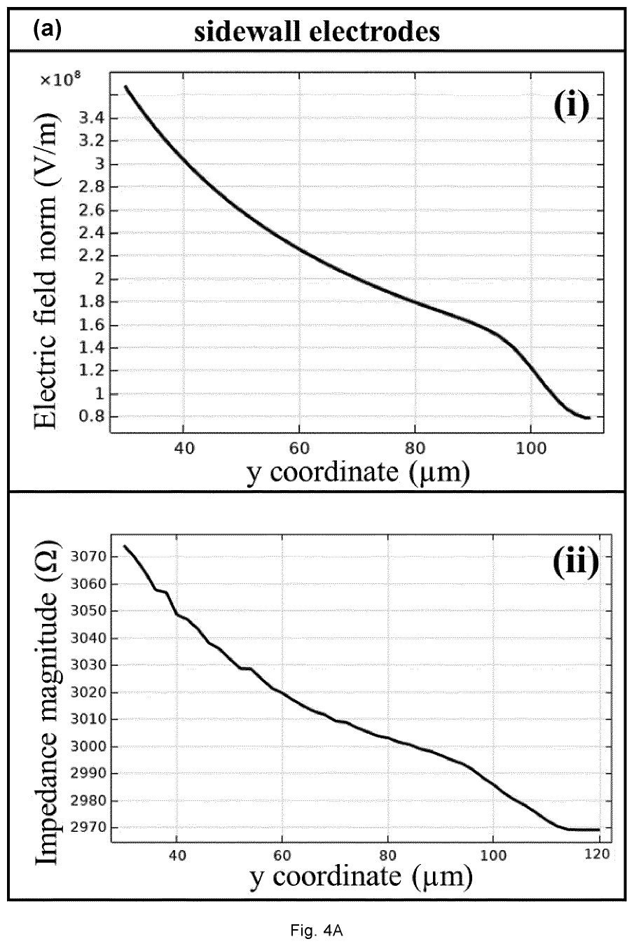

FIG. 4b shows the 3D electric field and impedance magnitude results of 3D electrodes (4b-iii and iv).

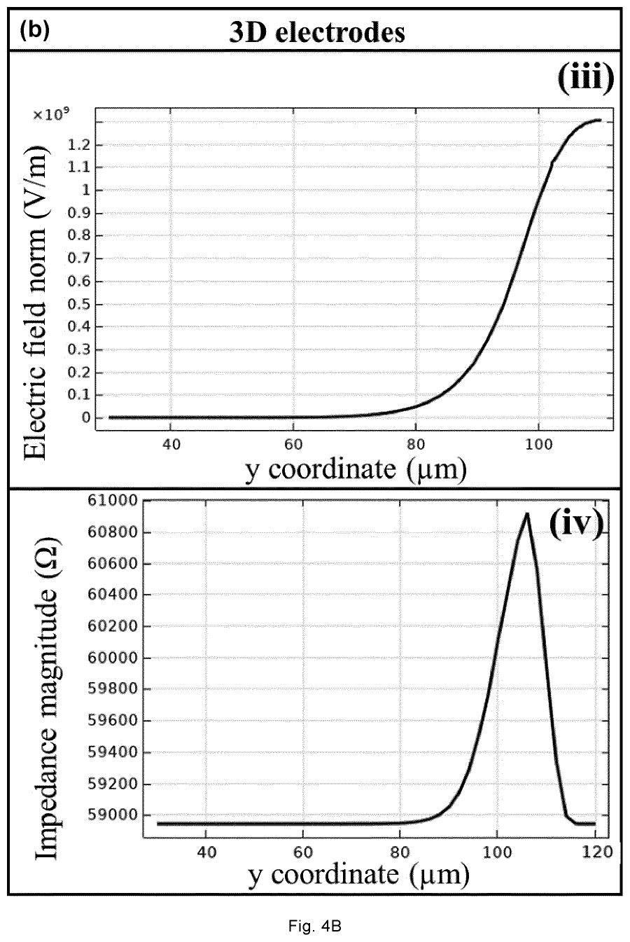

FIG. 5 shows schematic depictions of a side-wall (a) and 3D electrode integrated microsieve assembly (b).

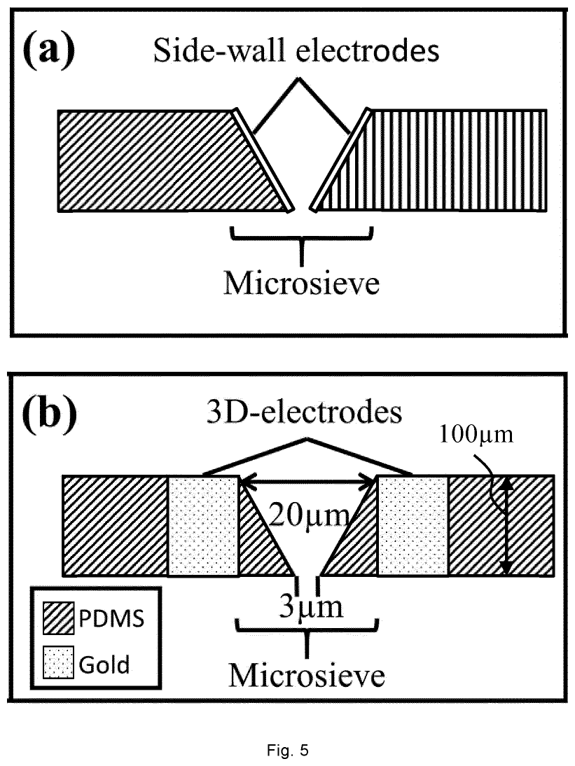

FIG. 6a shows results of electric field for side-wall (a) structures integrated on microsieves.

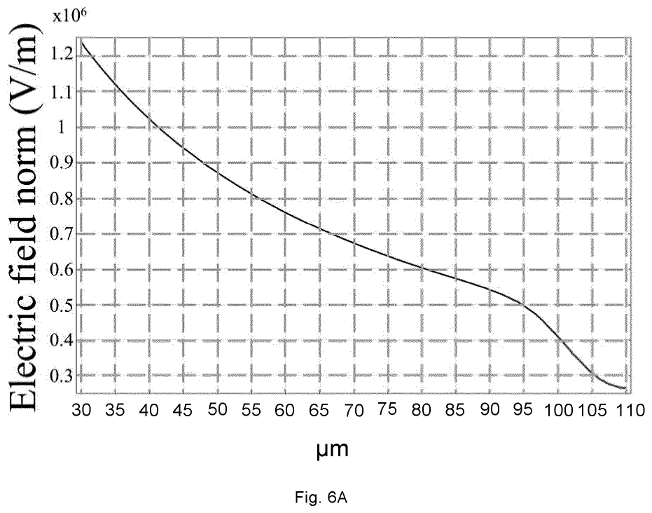

FIG. 6b shows results of electric field for 3D electrode (b) structures integrated in microsieves.

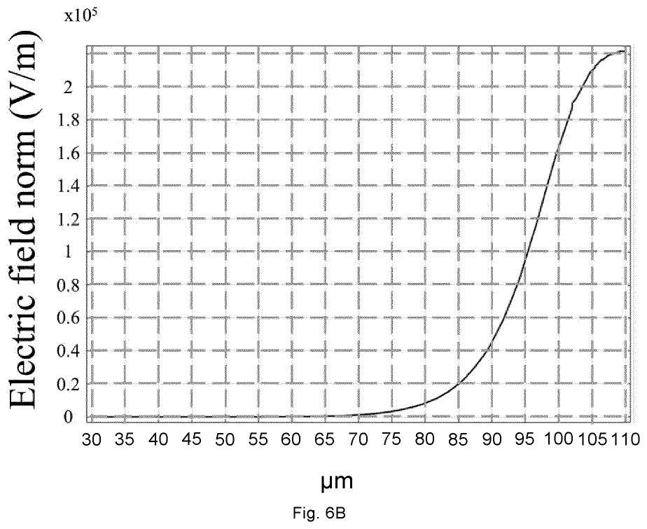

FIG. 7 shows impedance characteristics of 3D-electrodes of different conductivity solutions.

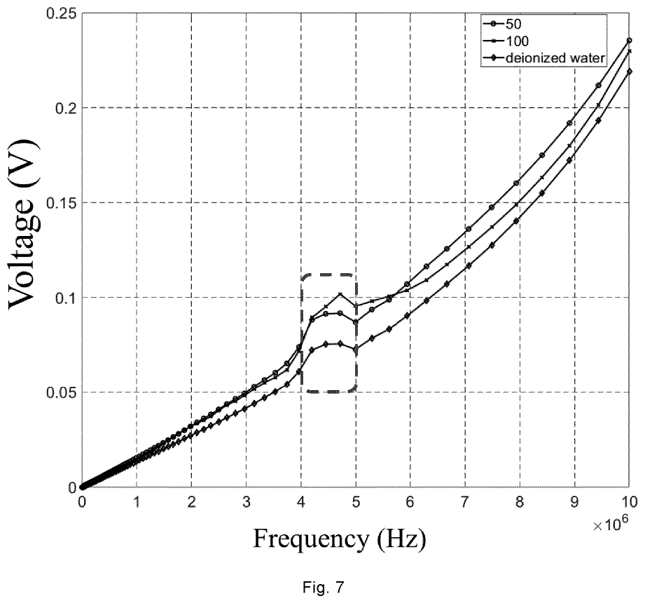

1. A device for detecting and/or characterizing one or more cells by the electrical properties of the cells, the device comprising at least one electrode integrated microsieve assembly, wherein the assembly comprises:
a. a microsieve arrangement, such as a microsieve array, comprising one or more micropores for retaining the cells; and
b. a substrate comprising one or more pairs of oppositely arranged first and second electrodes,
wherein the microsieve arrangement is connected to the substrate such that each of the one or more pairs of electrodes is configured to form an electric field in at least one micropore of the microsieve arrangement, and
wherein the first electrode is arranged in parallel to the second electrode.

2. The device of claim 1, wherein the microsieve arrangement is detachably connected to the substrate.

3. The device of claim 1, wherein the microsieve arrangement comprises one or more pairs of slots arranged such that each pair of first and second electrodes can be received into a respective pair of slots.

4. The device of claim 1, wherein the first slot is arranged at a distance of at most 75 μm, preferably at most 50 μm, more preferably about 20 μm from the second slot.

5. The device of claim 1, wherein the slots comprise a depth, a width and a height each arranged for receiving a respective electrode and/or wherein the slots have a depth of at most 70 μm, preferably at most 50 μm, more preferably about 20 μm and/or a width of at most 70 μm, preferably at most 50 μm, more preferably about 20 μm and/or a height of at most 200 μm, preferably about 100 μm.

6. A substrate comprising one or more pairs of oppositely arranged first and second electrodes for use in a device for detecting and/or characterizing one or more cells by the electrical properties of the cells, wherein the first electrode is arranged in parallel to the second electrode.

7. The substrate of claim 6, wherein the first electrode is arranged at a distance of at most 75 μm, preferably at most 50 μm, more preferably about 20 μm from the second electrode.

8. The substrate of claim 6, wherein the first and second electrodes have a height of at most 200 μm, preferably about 100 μm and/or a depth of at most 70 μm, preferably at most 50 μm, more preferably about 20 μm and/or a width of at most 70 μm, preferably at most 50 μm, more preferably about 20 μm.

9. The substrate of claim 6, wherein the electrodes are fabricated of a conductive material, preferably a metal, wherein the metal preferably is selected from the group consisting of copper, zinc, nickel, lead, mercury, silver, zinc, aluminum, gold, iron or alloy comprising any of the metals.

10. The substrate of claim 6, wherein the substrate comprises a hole, wherein the hole comprises a diameter of at most 10 μm, preferably about 3 μm, and wherein the hole is arranged such that it is between a pair of first and second electrodes.

11. A microsieve arrangement, such as a microsieve array, comprising one or more micropores for retaining one or more cells for use in an arrangement for detecting and/or characterizing one or more cells by the electrical properties of the cells.

12. The microsieve arrangement of claim 11, wherein the one or more micropores are in the form of a prism.

13. The microsieve arrangement of claim 11, wherein the one or more micropores comprise a form wherein the top-opening of the one or more micropores has a larger opening than the bottom-opening of the one or more micropores.

14. The microsieve arrangement of claim 11, wherein each of the micropores comprises a width, a depth and a height for retaining one or more cells and/or wherein the diameter of each one of the micropores is between at least 0 μm to 100 μm, preferably between about 2 μm and about 20 μm, and wherein the height is between about at least 0 μm to 200 μm, preferably about 5 μm to 100 μm, more preferably about 10 μm to 50 μm.

15. The microsieve arrangement of claim 11, wherein the one or more micropores are made from a material comprising a polymer.

16. The microsieve arrangement of claim 15, wherein the material has a permittivity constant of between about 1 and about 5, preferably about 2.75 and/or wherein the material comprising a polymer preferably is a silicon-based polymer, more preferably polydimethylsiloxane.

17-20. (canceled)
